(19) United States
(12) Patent Application Publication
(10) Pub. No.: US 2023/0183723 A1
(43) Pub. Date: Jun. 15, 2023

(54) MULTI-FUNCTIONAL GSH-RESPONSIVE SILICA NANOPARTICLES FOR DELIVERY OF BIOMOLECULES INTO PLANT CELLS

(71) Applicant: Wisconsin Alumni Research Foundation, Madison, WI (US)

(72) Inventors: Shaoqin GONG, Middleton, WI (US); Xuehua Zhong, Madison, WI (US); Yuyuan Wang, Madison, WI (US); Shuiming Qian, Madison, WI (US)

(21) Appl. No.: 17/926,113

(22) PCT Filed: May 18, 2021
(86) PCT No.: PCT/US2021/032946

§ 371 Date: Nov. 17, 2022

Related U.S. Application Data
(60) Provisional application No. 63/026,459, filed on May 18, 2020.

(51) Int. Cl. C12N 15/82; C08G 77/392; C12N 15/11; C12N 9/22

(52) U.S. Cl. CPC C12N 15/8206; C08G 77/392; C12N 15/8213; C12N 15/11; C12N 9/22; C12N 2310/20

(57) ABSTRACT

The present technology provides a nanoparticle that includes a silica network comprising crosslinked polysiloxanes, wherein the crosslinks comprise disulfide linkages, and the nanoparticle has a surface bearing charged functional groups and a surface potential of either less than −30 mV or greater than +30 mV, and wherein the nanoparticle has an average diameter of 20 nm to 60 n. The nanoparticles may be used to efficiently deliver biomolecules to plant cells, including polynucleic acids, proteins and complexes thereof (e.g., Cas9 RNP).

### CROSS REFERENCE TO RELATED APPLICATION

This application claims the benefit of and priority to U.S. Provisional Patent Application No. 63/026,459, filed on May 18, 2020, the entire contents of which is incorporated herein by reference in its entirety.

### STATEMENT OF GOVERNMENT SUPPORT

This invention was made with government support under 1844701 awarded by the National Science Foundation. The government has certain rights in the invention.

### FIELD

The present technology relates generally to the field of nanoplatform delivery systems. The delivery systems include a multi-functional GSH-responsive silica nanoparticles (SNPs) suitable for the delivery of biomolecules to plant cells. The nanoparticles have a highly charged surface and an average diameter of less than 60 nm, allowing them to efficiently deliver hydrophilic charged polynucleic acids, polypeptides (including proteins) and complexes of polypeptides and nucleic acids to plant cells. Methods of preparing and using the nanoparticles are also provided.

### BACKGROUND

Plants are critical sources of food, feed, fiber, fuel, and medicine for humans. Rapid climate changes, increased world population, together with a growing demand of carbon-neutral biofuels represent global challenges in meeting crop production needs. Genetic enhancement of crops via classical plant breeding approaches has been a significant means to meet crop improvement goals. Genetic engineering and genome editing have emerged as critical tools to accelerate progress and precision in crop functional genomics research and genetic enhancement. Current widely utilized plant genome editing systems are mainly based on transgene integration of exogenous DNA into the genome by Agrobacterium-mediated or microprojectile bombardment-mediated delivery of cargo DNA. While both methods have been successfully utilized in a number of plant species to generate transgenic plants for genome engineering, each has limited application due to low efficiency, genotype dependence, as well as complicated and time-consuming protocols. Agrobacterium or virus mediated transformation is commonly used but limited to a narrow range of plant species. Biolistic particle delivery cause plant tissue damage, specimen size limitation and require well trained personals.

The clustered regularly interspaced short palindromic repeats (CRISPR)-Cas9 systems are powerful tools for genome editing not only in animal cells but for plants as well. The Cas9/sgRNA ribonucleoprotein (RNP) can knock-out a target gene with high efficiency and specificity. Moreover, the combination of RNP and a DNA repair template (e.g., single-stranded donor oligonucleotide (ssODN) or donor polynucleic acid up to 2 kb) can achieve precise genome editing to incorporate sequences from the ssODN. However, safe and efficient delivery of RNP and RNP+donor DNA remains as a significant challenge for their potential application owing to their relatively large and complex structures. Similar to DNA and mRNA, unpackaged RNP and RNP+donor DNA are also susceptible to chemical degradation. Furthermore, in comparison to DNA and mRNA delivery, the delivery of protein/nucleic acid complexes such as RNP and RNP+donor DNA is even more challenging due to the mixed charges (e.g., positively charged Cas9 protein and negatively charged sgRNA and ssODN) and more sophisticated structures.

Despite the promise of CRISPR-Cas9, significant challenges remain in plant genome engineering and editing. One major hurdle has been the lack of an effective and efficient delivery system to deliver the editing tools into regenerative plant tissues and cells. Currently, Agrobacterium-mediated genetic transformation is the most commonly utilized and established CRISPR-Cas9 delivery approach in plants. In some plants such as Arabidopsis, a technique known as the flower dipping transformation method may be used to deliver CRISPR-Cas9 into plant cells. However, the flower dipping method only works on a very small number of plant species/genotypes and is not feasible for most plant species.

### SUMMARY OF THE INVENTION

The present technology provides a new nanoparticle for delivery of water-soluble biomolecules across the cell wall and membrane of plant cells to their interior. The new nanoparticle includes a silica network comprising crosslinked polysiloxanes, wherein the crosslinks comprise disulfide linkages, and the nanoparticle has a surface bearing charged functional groups and a surface potential of either less than −30 mV or greater than +30 mV. The nanoparticle may have an average diameter of 20 nm to 60 nm. The nanoparticles may include water-soluble biomolecules non-covalently bound to the nanoparticle. The water-soluble biomolecule is selected from the group consisting of a polynucleic acid, polypeptide, and a polynucleic acid/polypeptide complex.

The present technology also provides methods for delivering a water-soluble biomolecule into a plant cell comprising exposing the plant cell to an effective amount of a nanoparticle as described herein, wherein the nanoparticle includes the water-soluble biomolecule to be delivered.

### BRIEF DESCRIPTION OF THE DRAWINGS

FIGS. 1A-D illustrate the design, synthesis, and characterization of SNPs of the present technology. FIG. 1A schematically illustrates a non-limiting embodiment of the present SNPs for the delivery of various hydrophilic water-soluble biomolecules such as DNA, mRNA, and Cas9/sgRNA ribonucleoproteins (RNP). FIG. 1B schematically illustrates an exemplary synthesis of SNPs via a water-in-oil emulsion method. SNPs were collected by precipitation in acetone, centrifuged, and washed by ethanol and deionized water to remove all impurities. FIG. 1C shows a transmission electron microscopy micrograph of DNA-loaded SNPs of Example 1. FIG. 1D is a histogram showing the size distribution of DNA-loaded SNPs prepared according to Example 1 as measured by dynamic light scattering (DLS).

FIGS. 2A-C Size-dependent internalization of SNPs by plant cells. FIGS. 2A and 2B show DLS histograms of 50 nm and 120 nm SNPs discussed in Example 2. FIG. 2C shows photomicrographs of internalization of SNPs with different sizes by onion epithelial cells, imaged by CLSM 1 h post treatment.

FIGS. 3A-D. SNP delivery of GFP DNA into Arabidopsis mature leaves. FIG. 3A is a schematic diagram of SNP infiltration experiments described in Example 3. FIG. 3B schematically illustrates the putative intracellular trafficking pathway of SNPs in plant cells. FIGS. 3C and 3D provide representative confocal images showing the GFP protein intensity in Arabidopsis leaf cells 24 hours and 48 hours (respectively) post-infiltration with SNPs.

FIGS. 4A-C SNP delivery of GFP DNA into mature leaves of N. benthamiana and soybean. FIG. 4A provides representative confocal images showing the GFP protein in N. benthamiana leaf cells 48 hours post infiltration with SNPs. FIG. 4B provides representative confocal images showing the GFP protein in soybean mature leaf cells 24 and 48 hours post infiltration with SNPs. FIG. 4C provides representative confocal images showing overlapping expression of nuclei DNA stained with DAPI, innate GFP signal, and immunofluorescence stained GFP using antibodies again GFP after SNP transfection.

FIGS. 5A-B SNP delivery of GFP mRNA into plant leaves for transient expression. FIG. 5A shows representative confocal images of the GFP protein intensity in Arabidopsis leaf cells 24 and 48 hours post infiltration with SNPs. FIG. 5B shows representative confocal images of the GFP protein in N. benthamiana leaf cells 24 and 48 hours post infiltration with SNPs.

FIGS. 6A-B SNP-mediated CRISPR-Cas9 ribonucleoproteins (RNPs) delivery into Arabidopsis leaf cells. FIG. 6A shows representative images of GFP protein intensity in Arabidopsis leaf cells with SNP delivery of CRISPR-Cas9-GFP-sgRNA 2 days post infiltration. FIG. 6B shows detection of GFP DNA mutation efficiency by T7EI mismatch assays as discussed in Example 5.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; sulfates; phosphates; amines; N-oxides; hydrazines; hydrazides; hydrazones; azides (—N₃); amides; ureas; amidines; guanidines; enamines; imides; imines; nitro groups (—NO₂); nitriles (—CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, amidinealkyl, guanidinealkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above for alkyl.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic carbon-containing ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic carbon-containing ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent alkenyl groups are alkenylene groups, and so forth. Substituted groups having a single point of attachment to a compound or polymer of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, cycloalkyl, aryl, aralkyl, heterocyclylalkyl or heterocyclyl group as defined herein. Amido groups therefore include but are not limited to carbamoyl groups (—C(O)NH₂) (also referred to as “carboxamido groups”) and formamido groups (—NHC(O)H). In some embodiments, the amide is —NR⁷¹C(O)—(C₁₋₅ alkyl) and the group is termed “alkanoylamino.”

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, aryl aralkyl, heterocyclyl or heterocyclylalkyl group as defined herein. It will be understood that amidines may exist in protonated forms in certain aqueous solutions or mixtures and are examples of charged functional groups herein.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, cycloalkyl, aryl, aralkyl, heterocyclylalkyl or heterocyclyl group as defined herein. In some embodiments, the amine is NH₂, alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. It will be understood that amines may exist in protonated forms in certain aqueous solutions or mixtures and are examples of charged functional groups herein.

The term “carboxyl” or “carboxylate” as used herein refers to a —COOH group or its ionized salt form. As such, it will be understood that carboxyl groups are examples of charged functional groups herein.

The term “ester” as used herein refers to —COOR⁷⁰ and —C(O)O-G groups. R⁷⁰ is a substituted or unsubstituted alkyl, cycloalkyl, alkenyl, aryl, aralkyl, heterocyclylalkyl or heterocyclyl group as defined herein. G is a carboxylate protecting group. As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999), which can be added or removed using the procedures set forth therein and which is hereby incorporated by reference in its entirety and for any and all purposes as if fully set forth herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, aryl aralkyl, heterocyclyl or heterocyclylalkyl group as defined herein. It will be understood that guanidines may exist in protonated forms in certain aqueous solutions or mixtures and are examples of charged functional groups herein.

The term “hydroxyl” as used herein can refer to —OH or its ionized form, —O—. A “hydroxyalkyl” group is a hydroxyl-substituted alkyl group, such as HO—CH₂—.

The term “imidazolyl” as used herein refers to an imidazole group or the salt thereof. An imidazolyl may be protonated in certain aqueous solutions or mixtures, and is then termed an “imidazolate.”

The term “phosphate” as used herein refers to —OPO₃H₂ or any of its ionized salt forms, —OPO₃HR⁸⁴ or —OPO₃R⁸⁴R⁸⁵ wherein R⁸⁴ and R⁸⁵ are independently a positive counterion, e.g., Na⁺, K⁺, ammonium, etc. As such, it will be understood that phosphates are examples of charged functional groups herein.

The term “pyridinyl” refers to a pyridine group or a salt thereof. A pyridinyl may be protonated in certain aqueous solutions or mixtures, and is then termed a “pyridinium group”.

The term “sulfate” as used herein refers to —OSO₃H or its ionized salt form, —OSO₃R⁸⁶ wherein R⁸⁶ is a positive counterion, e.g., Na⁺, K⁺, ammonium, etc. As such, it will be understood that sulfates are examples of charged functional groups herein.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, and R⁸² are each independently a substituted or unsubstituted alkyl, cycloalkyl, alkenyl, aryl aralkyl, heterocyclyl or heterocyclylalkyl group as defined herein. In some embodiments the sulfide is an alkylthio group, —S-alkyl. R⁸³ includes H or, when the sulfonyl is ionized (i.e., as a sulfonate), a positive counterion, e.g., Na⁺, K⁺, ammonium or the like. As such, it will be understood that sulfonyls are examples of charged functional groups herein.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

As used herein, “Cas9 polypeptide” (also known as “Cas9”) refers to Cas9 proteins and variants thereof having nuclease activity, as well as fusion proteins containing such Cas9 proteins and variants thereof. The fused proteins may include those that modify the epigenome or control transcriptional activity. The variants may include deletions or additions, such as, e.g., addition of one, two, or more nuclear localization sequences (such as from SV40 and others known in the art), e.g., 1, 2, 3, 4, 5, 6, 7, 8, 9, 10 such sequences or a range between and including any two of the foregoing values. In some embodiments the Cas9 polypeptide is a Cas9 protein found in a type II CRISPR-associated system. Suitable Cas9 polypeptides that may be used in the present technology include, but are not limited to Cas9 protein from Streptococcus pyogenes (Sp. Cas9), F. novicida, S. aureus, S. thermophiles, N. meningitidis, and variants thereof. In some embodiments, the Cas9 polypeptide is a wild-type Cas9, a nickase, or comprises a nuclease inactivated (dCas9) protein. In some embodiments, the Cas9 polypeptide is a fusion protein comprising dCas9. In some embodiments, the fusion protein comprises a transcriptional activator (e.g., VP64), a transcriptional repressor (e.g., KRAB, SID) a nuclease domain (e.g., FokI), base editor (e.g., adenine base editors, ABE), a recombinase domain (e.g., Hin, Gin, or Tn3), a deaminase (e.g., a cytidine deaminase or an adenosine deaminase) or an epigenetic modifier domain (e.g., TET1, p300). In some embodiments, the Cas9 polypeptide includes variants with at least 85% sequence identity, at least 90% sequence identity, at least 95% sequence identity, or even 96%, 97%, 98%, or 99% sequence identity to the wild type Cas9. Accordingly, a wide variety of Cas9 polypeptides may be used as formation of the nanoparticle is not sequence dependent so long as the Cas9 polypeptide can complex with nucleic acids and the resulting RNP may associate with the other constituents of the present nanoparticles. Other suitable Cas9 polypeptides may be found in Karvelis, G. et al. “Harnessing the natural diversity and in vitro evolution of Cas9 to expand the genome editing toolbox,” Current Opinion in Microbiology 37: 88-94 (2017); Komor, A. C. et al. “CRISPR-Based Technologies for the Manipulation of Eukaryotic Genomes,” Cell 168:20-36 (2017); and Murovec, J. et al. “New variants of CRISPR RNA-guided genome editing enzymes,” Plant Biotechnol. J. 15:917-26 (2017), each of which is incorporated by reference herein in their entirety.

“Molecular weight” as used herein with respect to polymers refers to number-average molecular weights (Mₙ) and can be determined by techniques well known in the art including gel permeation chromatography (GPC). GPC analysis can be performed, for example, on a D6000M column calibrated with poly(methyl methacrylate) (PMMA) using triple detectors including a refractive index (RI) detector, a viscometer detector, and a light scattering detector, and N,N′-dimethylformamide (DMF) as the eluent. “Molecular weight” in reference to small molecules and not polymers is actual molecular weight, not number-average molecular weight.

“Organosilica network” refers to a network containing crosslinked polysiloxane polymers. Polysiloxanes of the present technology comprise repeating silicon-containing substructures of which a fraction (e.g., about 0.01 mol % to about 90 mol %, such as 0.1, 1, 5, 10, 20, 30, 40, 50, 60, 70, 80, or 90 mol %, or a range between and including any two of the foregoing values, including about 0.1 mol % to about 90 mol %, about 1 mol % to about 80 mol %, or about 10 mol % to about 90 mol %) of the repeating silicon-containing substructures include one or more crosslinks to another polysiloxane chain. The crosslinks may include disulfide linkages (—S—S—) and or siloxy ether linkages (e.g., —Si—O—Si—). The organosilica network may include silicon atoms with two polymeric attachment points (i.e., the silicon forms part of a linear polysiloxane chain) and/or three and/or four polymeric attachment points (i.e., crosslinks to polysiloxane chains

A “polysiloxane” as used herein refers to a linear or branched polymer comprising repeating silyloxy subunits attached to each other through Si—O—Si (silyl ether) linkages. Polysiloxanes may be homopolymers or copolymers, including random copolymers of more than one type of siloxy subunit.

A “cell penetrating peptide” (CPP), also referred to as a “protein transduction domain” (PTD), a “membrane translocating sequence,” and a “Trojan peptide”, refers to a short peptide (e.g., from 4 to about 40 amino acids) that has the ability to translocate across a cellular membrane to gain access to the interior of a cell and to carry into the cells a variety of covalently and noncovalently conjugated cargoes, including the present nanoparticles and the water-soluble biomolecules. CPPs are typically highly cationic and rich in arginine and lysine amino acids. Examples of such peptides include TAT cell penetrating peptide (GRKKRRQRRRPQ); MAP (KLALKLALKALKAALKLA); Penetratin or Antenapedia PTD (RQIKWFQNRRMKWKK); Penetratin-Arg: (RQIRIWFQNRRMRWRR); antitrypsin (358-374): (CSIPPEVKFNKPFVYLI); Temporin L: (FVQWFSKFLGRIL-NH2); Maurocalcine: GDC(acm) (LPHLKLC); pVEC (Cadherin-5): (LLIILRRRIRKQAHAHSK); Calcitonin: (LGTYTQDFNKFHTFPQTAIGVGAP); Neurturin: (GAAEAAARVYDLGLRRLRQRRRLRRERVRA); Penetratin: (RQIKIWFQNRRMKWKKGG); TAT-HA2 Fusion Peptide: (RRRQRRKKRGGDIMGEWGNEIFGAIAGFLG); TAT (47-57) Y(GRKKRRQRRR); SynB1 (RGGRLSYSRRRFSTSTGR); SynB3 (RRLSYSRRRF); PTD-4 (PIRRRKKLRRL); PTD-5 (RRQRRTSKLMKR); FHV Coat-(35-49) (RRRRNRTRRNRRRVR); BMV Gag-(7-25) (KMTRAQRRAAARRNRWTAR); HTLV-II Rex-(4-16) (TRRQRTRRARRNR); HIV-1 Tat (48-60) or D-Tat (GRKKRRQRRRPPQ); R9-Tat (GRRRRRRRRRPPQ); Transportan (GWTLNSAGYLLGKINLKALAALAKKIL chimera); SBP or Human P1 (MGLGLHLLVLAAALQGAWSQPKKKRKV); FBP (GALFLGWLGAAGSTMGAWSQPKKKRKV); MPG (ac-GALFLGFLGAAGSTMGAWSQPKKKRKV-cya (wherein cya is cysteamine)); MPG(ANLS) (ac-GALFLGFLGAAGSTMGAWSQPKSKRKV-cya); Pep-1 or Pep-1-Cysteamine (ac-KETWWETWWTEWSQPKKKRKV-cya); Pep-2 (ac-KETWFETWFTEWSQPKKKRKV-cya); Periodic sequences, Polyarginines (RxN (4<N<17) chimera); Polylysines (KxN (4<N<17) chimera); (RAca)6R; (RAbu)6R; (RG)6R; (RM)6R; (RT)6R; (RS)6R; R10; (RA)6R; and R7.

A “dye” refers to small organic molecules having a molecular weight (actual, not number average) of 2,000 Da or less or a protein which is able to emit light. Non-limiting examples of dyes include fluorophores, chemiluminescent or phosphorescent entities. For example, dyes useful in the present technology include but are not limited to cyanine dyes (e.g., Cy2, Cy3, Cy5, Cy5.5, Cy7, and sulfonated versions thereof), fluorescein isothiocyanate (FITC), ALEXA FLUOR© dyes (e.g., ALEXA FLUOR© 488, 546, or 633), DYLIGHT© dyes (e.g., DYLIGHT© 350, 405, 488, 550, 594, 633, 650, 680, 755, or 800) or fluorescent proteins such as GFP (Green Fluorescent Protein).

The present technology provides silica nanoparticles (SNPs) suitable for delivering water-soluble biomolecules into plant cells. Each nanoparticle includes a silica network comprising crosslinked polysiloxanes, wherein the crosslinks include disulfide linkages, and the nanoparticle has a surface bearing charged functional groups and a surface potential of either less than −30 mV or greater than +30 mV, and wherein the nanoparticle has an average diameter of 20 nm to 60 nm. In any embodiments, each nanoparticle includes a water-soluble biomolecule. Such biomolecules are encapsulated at least in part within the SNP.

In any embodiments of the nanoparticle, the polysiloxanes comprise a plurality of siloxy subunits having the structure

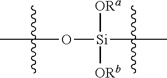

and/or the structure

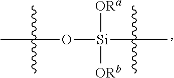

wherein R^{a} and R^{b} at each occurrence in the polysiloxane are independently selected from a bond to a Si of another polysiloxane chain or C₁₋₆ alkyl groups, and R^{c} is selected from C₂₋₆ alkenyl groups. In any embodiments, the polysiloxanes comprising the plurality of siloxy subunits having the structure

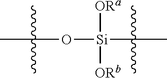

may include a first portion of siloxy subunits wherein R^{a} and R^{b} are independently selected from C₁₋₆ alkyl groups, and a second portion of siloxy subunits wherein one of R^{a} and R^{b} is independently selected from C₁₋₆ alkyl groups at each occurrence, and one of R^{a} and R^{b} is a bond to a Si of another polysiloxane chain. It will be appreciated that when R^{a} or R^{b} is a bond to a Si of another polysiloxane chain, the siloxysubunit is branched, forming a crosslink to another polysiloxane chain. In any embodiments, the plurality of siloxy subunits may be derived from tetraethoxysilane and/or triethoxyvinylsilane, i.e., these monomers are precursors which polymerize to form the siloxy subunits. In any embodiments, the polysiloxanes further include a plurality of siloxy subunits bearing imidazolyl groups such as, but not limited to N-(3-(triethoxysilyl)propyl)-1H-imidazole-2-carboxamide (TESPIC).

Silica nanoparticles of the present technology are multifunctional. For example, the polysiloxanes that make up the silica network include a plurality of crosslinking siloxy subunits having the structure

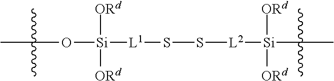

wherein L¹ and L² at each occurrence in the polysiloxanes are independently a C₁₋₆ alkylene group; R^{d} at each occurrence in the polysiloxanes is the same or different and is independently selected from a bond to another polysiloxane chain or C₁₋₆ alkyl groups. The disulfide bonds are sensitive to the levels of glutathione (GSH) naturally found in cells. While not wishing to be bound by theory, when SNPs enter a plant cell, the GSH in the cell is believed to reduce the disulfide bonds in the silica network, causing the silica network to fall apart and release any encapsulated water-soluble biomolecule into the cytosol of the plant cell.

In the present technology, the surface of the SNPs is highly charged due to the present of ionizable functional groups on the SNP surface and/or in the SNP surface layer. For example, in any embodiments, the polysiloxanes of the silica network may comprise a plurality of siloxy subunits having the structure

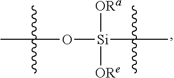

wherein R^{a} at each occurrence in the polysiloxane is a bond to Si from another polysiloxane chain or a C₁₋₆ alkyl group, and R^{e} at each occurrence is a C₁₋₆ alkyl group substituted with a charged functional group. The charged functional groups may include positively or negatively charged groups, or ionizable functional groups that provide positively or negatively charged groups, or mixtures thereof. Where the charged functional groups include mixtures of both negatively and positively charged groups, the surface potential must still be either less than −30 mV or greater than +30 mV.

In any embodiments, the charged functional groups may include positively charged groups. In any embodiments, the charged functional groups include an ionizable group selected from amine, amidine, guanidine, pyridinyl or combinations of two or more thereof. For example, R^{e} may be an amino-(C₂₋₄ alkyl) group such as an amino propyl group. The charged functional groups may also be positively charged groups on a cationic polymer or CPP; e.g., the surface of the nanoparticle may include a cationic polymer or a CPP. For example, the cationic polymer may be selected from the group consisting of polyethyleneimine (PEI), polylysine, polyarginine, and polyamidoamine (PAMAM). In any embodiments, the CPP may be selected from any of those disclosed herein.

In any embodiments, the charged functional groups may include negatively charged groups. In any embodiments, the charged functional groups may include ionizable functional groups selected from carboxyl, sulfonyl, sulfate, phosphate, or combinations thereof. In any embodiments, R^{e} may be a carboxyl-(C₂₋₄ alkyl) group. The charged functional groups may also include negatively charged groups on an anionic polymer; e.g., the surface of the nanoparticle may include an anionic polymer. In any embodiments, the anionic polymer may be selected from the group consisting of poly(glutamic acid) and poly(acrylic acid).

In any embodiments, where the charged functional groups are part of a polymer (e.g., polycation or polyanion), the polymer may have a Mn of about 1,000 to about 50,000 Da. For example, the polycation or polyanion may have a Mn of about 1,000, about 2,000, about 3,000, about, 4,000, about 5,000, about 7,500, about 10,000, about 15,000, about 20,000, about 30,000, about 40,000, about 50,000 Da or a value within a range between and including any two of the foregoing values. For example, the polycation or polyanion may have a Mn of about 2,000 to about 10,000 Da.

The surface potential (zeta potential) of the SNP may be either equal to or less than −30 mV or equal to or greater than +30 mV. Nanoparticle surface potential may be measured by DLS in an applied electric field at any suitable voltage (e.g., 40 V; the measured surface potential will be independent of the exact voltage used) at 0.1 mg/mL, pH 7.4, 25° C. In any embodiments, the charged functional groups are positively charged groups and the surface potential is ≥+30 mV, ≥+35 mV, ≥+40 mV. In any such embodiments, the surface potential may be ≤+60 mV, +50 mV, or ≤+40 mV. In any embodiments, the surface potential may be a range between and including any two of the foregoing values, e.g., from equal to or greater than +30 mV to equal to or less than +60 mV, or from ≥+30 mV to ≤+40 mV. Likewise, in any embodiments, the charged functional groups are negatively charged groups, and the surface potential may be ≤−30 mV, ≤−35 mV, or ≤−40 mV. In any such embodiments, the surface potential may be ≥−60 mV, ≥−50 mV, or ≥−40 mV. In any embodiments, the surface potential may be a range between and including any two of the foregoing values, e.g., from ≤−30 mV to ≥−60 mV, or from ≤−30 mV to ≥−40 mV.

The present SNPs may be roughly sphere-shaped or may have a more elongated shape. Nevertheless, the “average diameter” of the present SNPs means the average hydrodynamic diameter and ranges from 20 nm to 60 nm. This small size facilitates the ability of the SNPs to cross the plant cell wall and membrane. Thus, the present SNPs may have an average hydrodynamic diameter of 20, 30, 40, 50, or 60 nm or a range between and including any two of the foregoing values. In any embodiments herein, they may have an average hydrodynamic diameter of 20 to 50 nm or even 30 to 50 nm.

In any embodiments, the present SNPs further include a water-soluble biomolecule non-covalently bound to the nanoparticle. For example, the water-soluble biomolecule may be encapsulated by the SNP and/or electrostatically bound to the SNP. In any embodiments the majority of the water-soluble biomolecule is encapsulated within the SNP. As used herein, “water-soluble” refers to a solubility of at least 1 mg/ml in water at pH 7 and 25° C. The water-soluble biomolecule may be a polynucleic acid, polypeptide, or a polynucleic acid/polypeptide complex, e.g., DNA, RNA, an enzyme, or a ribonucleoprotein complex (RNP). In any embodiments, the water-soluble biomolecule may be selected from the group consisting of plasmid DNA (pDNA), single-stranded donor oligonucleotide (ssODN), complementary (cDNA), messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof. In certain embodiments, the water-soluble biomolecule may be selected from the group consisting of Cas9 RNP, RNP+ssODN where ssODN serves as a repair template, RNP+donor DNA up to 2 kb, and other Cas9-based protein/nucleic acid complexes. It will be appreciated that with the present nanoparticles, Cas9 or RNP need not be conjugated to any repair template as either may simply be mixed with the desired polynucleic acid instead during the nanoparticle formation process. NLS peptides may be used to direct water-soluble biomolecule to the nucleus if desired. For example, polynucleic acids as described herein as well as proteins such as Cas9 or RNP+ donor DNA complexes may be covalently tagged with NLS peptides using techniques well known in the art.

In any embodiments, the water-soluble biomolecule may be tagged with a dye as described herein. Alternatively, a dye and/or an imaging agent may be attached to the organosilica network. The dye or imaging agent may be attached to the organosilica network via bonds to amino groups in the organosilica network. By way of a non-limiting example, the bonds may be amide bonds, N—C bonds, imino bonds and the like.

The present SNPs may have a biomolecule loading content of from about 1 wt % to about 20 wt %, e.g., about 1 wt %, about 2 wt %, about 3 wt %, about 4 wt %, about 5 wt %, about 6 wt %, about 7 wt %, about 8 wt %, about 9 wt %, about 10 wt %, about 12 wt %, about 14 wt %, about 15 wt %, about 16 wt %, about 18 wt %, or about 20 wt %, or a range between and including any two of the foregoing values. Thus, in any embodiments, the biomolecule loading content of the SNP may be, e.g., from about 2 wt % to 20 wt %, about 5 wt % to about 15 wt %, or about 8 or 9 wt % to about 10 wt %. Loading efficiency of the present SNPs with biomolecules is high. In any embodiments, the loading efficiency may be greater than 80%, greater than 85%, or even greater than 90%, e.g., 80%, 85%, 90%, 95%, 99% or a range between and including any two of the foregoing values.

In another aspect, the present technology provides methods of making the silica nanoparticles described herein. The methods include forming a nanoparticle comprising an organosilica network as described herein by combining an aqueous solution, optionally containing the water-soluble biomolecules and a solution of organosilica network precursors in an immiscible organic solvent, and forming an emulsion, e.g., by rapid stirring. Optionally, a catalyst such as a base is added to facilitate the polymerization of the organosilica network precursors to form the organosilica network. After the initial polymerization, cationic or anionic siloxy precursors may be added to the mixture to polymerize with the nascent nanoparticles and provide the required surface potential. The cationic and anionic precursors may be further functionalized before or after being added to the nanoparticle mixture. The organosilica network precursors may include various tetraalkoxysilanes and organosiloxy disulfide monomers. Optionally trialkoxy alkyl silanes or trialkoxy alkenyl silanes may be used in place of or in addition to the tetraalkoxysilane. The water-soluble biomolecule may selected from any of the biomolecules disclosed herein. The emulsion may be formed from any suitable organic solvents (including, e.g., alkanes, cycloalkanes, alcohols and non-ionic detergents and mixtures of any two or more thereof) and water. In any embodiment, the emulsion may include hexanol, cyclohexane, Triton X-100 (polyethylene glycol p-(1,1,3,3-tetramethylbutyl)-phenyl ether) and water. In any embodiments, the emulsion may be formed by any suitable methods such as rapid stirring, shaking, vortexing, and sonication. The emulsion must be agitated sufficiently vigorously to form nanoparticles of the size desired for the present technology, i.e. <60 nm when carrying the water-soluble biomolecule. The molar ratio of disulfide-containing crosslinker to the total organosilica precursors may range from 20 mol % to 80 mol %, including for example, 20 mol %, 30 mol %, 40 mol %, 50 mol %, 60 mol %, 70 mol %, 80 mol % or a range between and including any two of the foregoing values. The molar ratio of siloxy amines (or other charged functional groups) to the total organosilica precursors may range from 10 mol % to 50 mol %. In any embodiments, the present methods may further include attaching a CPP, polycation, or polyanion to the surface of the SNP. The polycations and polyanions typically have a reactive group such as an electrophile or active ester or the like which can react with, e.g., a nucleophilic group on the organosilica network such as, but not limited to amino groups. Other amide-bond forming methods or click chemistry may be used join the CPP, polycation or polyanion to the nanoparticle. Alternatively, the CPP, polycation, or polyanion can simply be adsorbed to the surface of the nanoparticle via electrostatic interactions. The nanoparticles thus formed may be precipitated from solution with a suitable organic solvent, e.g., acetone.

In another aspect, the present technology provides methods of delivering a water-soluble biomolecule to a target plant cell for any suitable purpose, e.g., gene editing. The methods include exposing the targeted cell to an effective amount of any of the herein-described nanoparticles. By an effective amount is meant an amount sufficient to produce a detectable or measurable amount of infiltration of the SNP into the target plant cell and/or produce a detectable or measurable effect in said plant cell. The methods include both in vitro and in vivo methods. For example, the methods may include exposing an effective amount of any of the herein-described nanoparticles to plant tissue culture. In any embodiments, the plant may be exposed to the SNP via a leaf, stem, root, flower, or other part of the plant. In any embodiments, the water-soluble biomolecule is any of those described herein, including but not limited to DNA, pDNA, mRNA, siRNA, Cas9 RNP, RNP+donor nucleic acids.

The examples herein are provided to illustrate advantages of the present technology and to further assist a person of ordinary skill in the art with preparing or using the nanoparticles compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

### Materials and General Procedures

Materials. Tetraethyl orthosilicate (TEOS), tetrahydrofuran (THF), Triton X-100, acetone, ethanol, and ammonia (30% in water) were purchased from Fisher Scientific, USA. Hexanol, cyclohexane, and (3-aminopropyl)triethoxysilane (APTES), were bought from Tokyo Chemical Industry Co., Ltd., USA. Bis[3-(triethoxysilyl)propyl]-disulfide (BTPD) was purchased from Gelest, Inc., USA. Nuclear localization signal (NLS)-tagged Streptococcus pyogenes Cas9 nuclease (sNLS-SpCas9-sNLS) was provided by Aldevron, USA. Guide RNA components, crRNA (target sequence: TTCAAGAGCGCCATGCCTG) and tracrRNA were purchased from Integrated DNA Technologies, Inc. ATTO550 tagged guide RNA for SNP uptake studies was purchased from Integrated DNA Technologies, Inc. Anti-GFP antibodies were purchased from Sigma (11814460001).

Plants. Arabidopsis thaliana ecotype Columbia-0 (Col-0) was used as a wild-type. The transgenic Arabidopsis line expressing p35S::GFP was generated by agrobacterium-mediated floral dip into Col-0. T3 homozygous plants were used for the NP studies. Seeds were sown in soil and kept at 4° C. for 2 days before transferring to 16-hours light using a set of fluorescent lamps (Philips, 40 W) at around 100 μmol·m-2.s-1 and 8-hours dark (long day) at 22° C.

### Example 1—Preparation and Characterization of GSH-Responsive Silica Nanoparticles (SNPs)

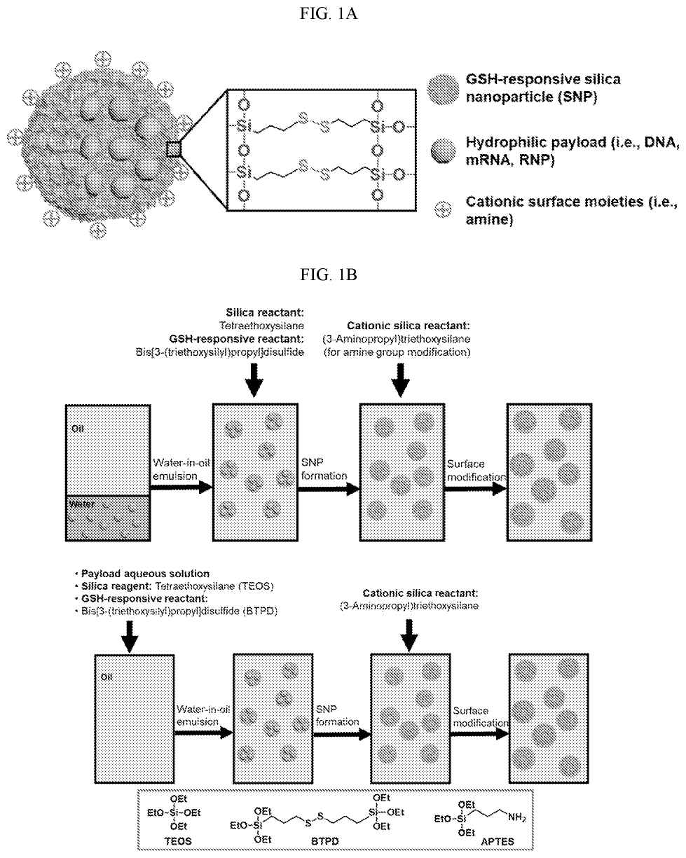

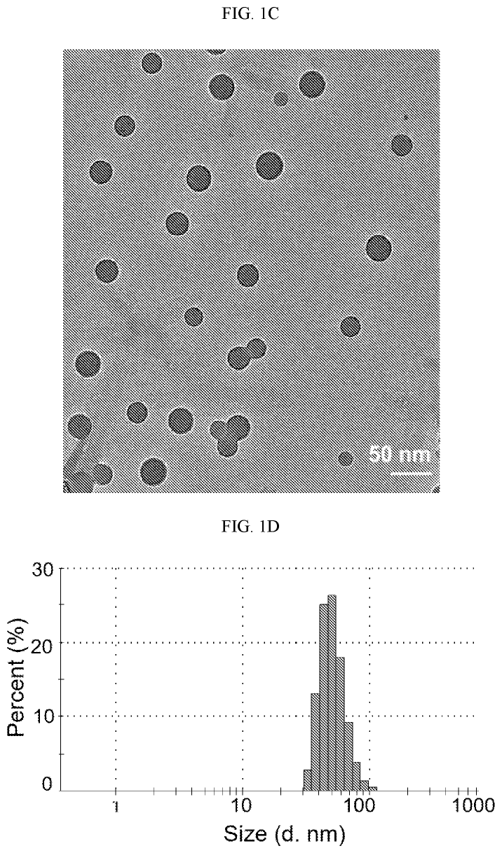

Preparation. FIG. 1B depicts schematically how an illustrative embodiment of SNPs of the present technology (FIG. 1A) were synthesized by a water-in-oil emulsion method. Triton X-100 (1.75 ml) and hexanol (1.75 ml) were dissolved in cyclohexane (7.5 ml) to form an organic solution to serve as the organic phase in a water-in-oil emulsion. Separately, 30 μl of a 5 mg/ml aqueous solution of desired water-soluble biomolecule (e.g., DNA, mRNA and RNP) were mixed with 4 μl of TEOS and 6 μl of BTPD. After shaking, this mixture was added to 1.1 ml of the organic solution, and the water-in-oil microemulsion was formed by vortexing for 1 min. Under vigorous stirring, an aliquot of 5 μl of 30% aqueous ammonia solution was added and the water-in-oil microemulsion was stirred at room temperature for 4 h to obtain unmodified SNPs. The size of the SNPs can be controlled by the stirring rate. For example, to obtain 50 nm SNPs, the stirring rate was 1500 rpm; for 120 nm SNP synthesis, the stirring rate was 600 rpm. To prepare positively charged SNPs, the as-prepared SNP was modified with amine groups by addition of APTES to the microemulsion, and the mixture was stirred overnight at room temperature. To purify the SNPs, 1.5 mL of pure acetone was added in the microemulsion in order to precipitate the SNPs and the material was recovered by centrifugation, washing twice with ethanol, and three times with water.

SNP characterization. The hydrodynamic diameter and zeta potential of the SNPs were characterized by a dynamic light scattering (DLS) spectrometer (Malvern Zetasizer Nano ZS) at a 900 detection angle with a concentration of 0.1 mg/ml. The morphologies of SNPs were characterized by transmission electron microscopy (TEM, Tecnai 12, Thermo Fisher, USA). As shown in FIG. 1C, the SNPs exhibited spherical morphologies with uniform sizes around 30-40 nm. Overall, the cargo-loaded SNPs showed hydrodynamic diameters between 40-50 nm (FIG. 1D). Table 1 shows the hydrodynamic diameter and and zeta-potential of several cargo-loaded SNPs. The measured zeta-potential shows that amine group modification of SNPs resulted in highly positive surface charge regardless of the water-soluble biomolecule, indicating that the SNP is suitable for nucleic acid and RNP delivery.

SNP Loading content/loading efficiency. To calculate the loading content and loading efficiency of the water-soluble biomolecules in the SNPs, 1 mg/ml of SNP stock solution with different water-soluble biomolecules were prepared. Thereafter, 10 μL of SNP was incubated with 0.1 M GSH aqueous solution (40 μl) for 1 h to allow for complete dissociation of SNPs. The RNP loading contents/efficiencies were measured via a bicinchoninic acid assay (BCA assay, Thermo Fisher, USA). DNA and mRNA loading contents/efficiencies were evaluated using a NanoDrop One (Thermo Fisher, USA).

The loading content and loading efficiency of different water-soluble biomolecules are summarized in Table 1. The SNPs showed similar loading content (>9.5 wt %) and loading efficiency (91-94%) with different water-soluble biomolecules including DNA, mRNA, and RNP. The uniformly high loading content and efficiency regardless of water-soluble biomolecule type can be attributed to the water-in-oil emulsion method that confines the water-soluble biomolecules within the water droplet where the silica network forms simultaneously, resulting in the complete encapsulation of the water-soluble biomolecule.

TABLE 1
Summary of loading content and efficiency of
different water-soluble biomolecules by SNPs.

| | Hydrodynamic | Zeta- | Loading | Loading |
|---|---|---|---|---|
| Water-soluble | diameter | potential | content | efficiency |
| biomolecule | (nm) | (mV) | (wt %) | (%) |
| DNA | 47 | 33 | 9.5 | 91 |
| mRNA | 45 | 35 | 9.6 | 92 |
| RNP | 41 | 37 | 9.8 | 94 |

### Example 2—SNP Uptake by Onion Cells

Guide RNA labeled with the ATTO550 fluorescence tag was complexed with Cas9 protein to form ATTO550-RNP, and encapsulated into SNPs of different sizes (i.e., 50 nm and 120 nm) according to the protocol of Example 1. Fresh onions were purchased locally, and epidermal cells were peeled off from the abaxial surface. The epidermal cells were then placed on a microscope glass slide and treated with ATTO550-RNP-loaded SNPs with a fixed RNP concentration of 50 μg/ml. One-hour post-treatment, the onion epidermal cells were rinsed with water, and then stained with nucleus marker Hoechst 33342 (10 μg/ml). Samples were then covered with a glass cover slip and imaged under a confocal laser scanning microscope.

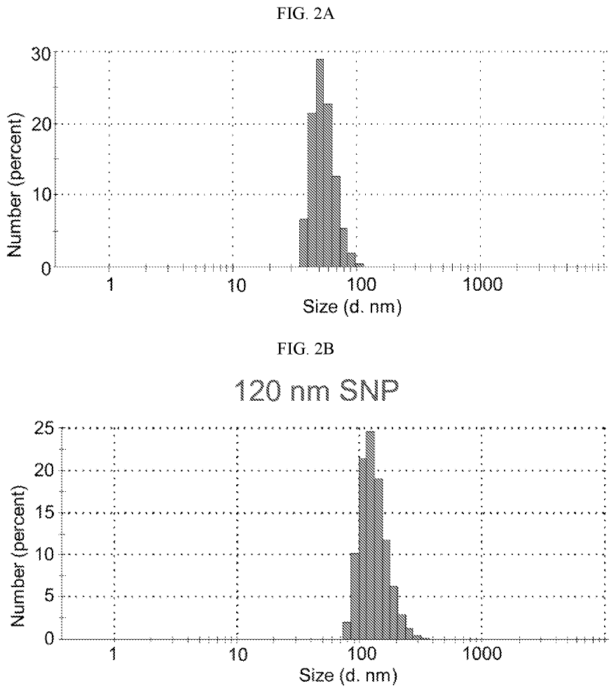

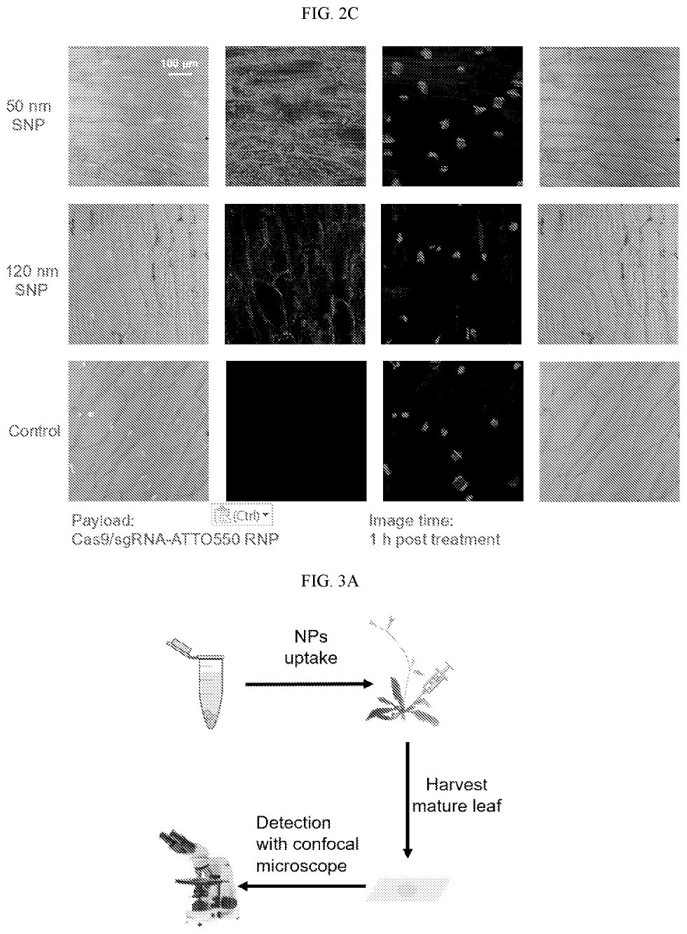

Results. To study the size effect of SNPs on cellular uptake efficacy, two RNP-loaded SNPs were prepared with the same surface charge but different sizes (i.e., 50 nm and 120 nm; FIGS. 2A and 2B). The water-soluble biomolecule RNP was tagged with a fluorescence dye, ATTO550 on the guide RNA. Uptake of SNPs with different sizes in onion epithelial cells was studied by confocal laser scanning microscopy (CLSM), as shown in FIG. 2C. After 1 h treatment, 60 nm SNPS were readily internalized by onion epithelial cells; in contrast, 120 nm SNP treated cells exhibited significantly lower ATTO550 fluorescence, with signals mainly observed in the region around the cell peripheral (i.e., cell wall). These results indicate that the plant cell wall plays a dominant role in excluding larger SNPs. The SNPs with a hydrodynamic size of less than 50 nm were observed to penetrate the cell wall and enter the plant cells.

### Example 3—SNP Infiltration into the Plant Leaf Cells—Part 1

Three-week old Arabidopsis thaliana and N. benthamiana or ten-day old soybean Williams genotype plants were used for SNP infiltration. BD tuberculin syringe (Fisher Scientific, USA) filled with SNP was used to gently inject the SNPs into the leaf from the abaxial side without penetrating the adaxial side. The infiltrated plants were grown at 22° C. under long-day condition at 22° C.

GFP detection using confocal laser scanning microscope. Plant leaves infiltrated with SNPs containing GFP DNA, mRNA, and RNPs were imaged with Olympus FluoView 1000 Confocal Laser Scanning Microscope (Olympus America, Inc., Center Valley, Pa., United States). A fully automated Olympus IX81 inverted microscope with a 10× UPlanSApo objective (NA 0.40) was used to measure the GFP fluorescence intensity (green) from a single confocal plane as well as the corresponding transmitted laser light brightfield image (gray). GFP fluorescence was excited using the 488 nm Argon gas laser line and detected using a 505-525 nm band pass emission filter.

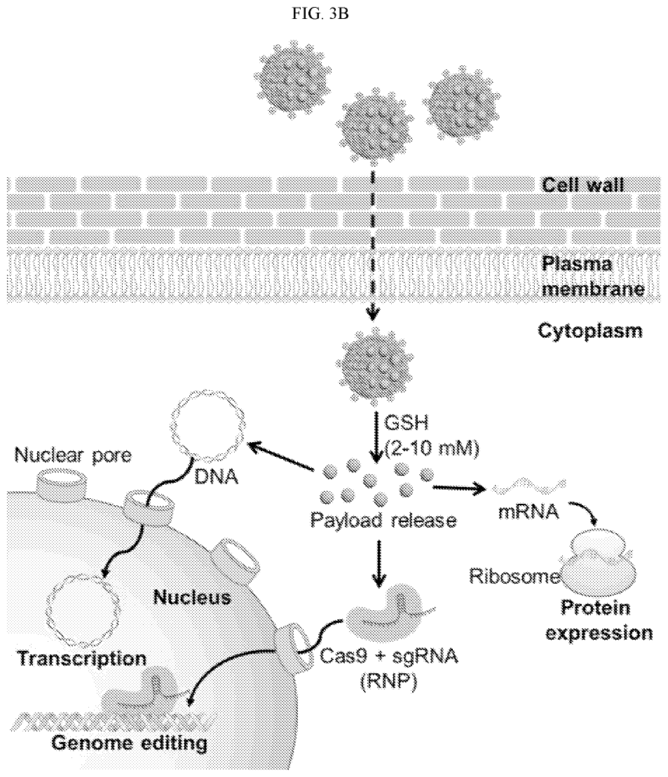

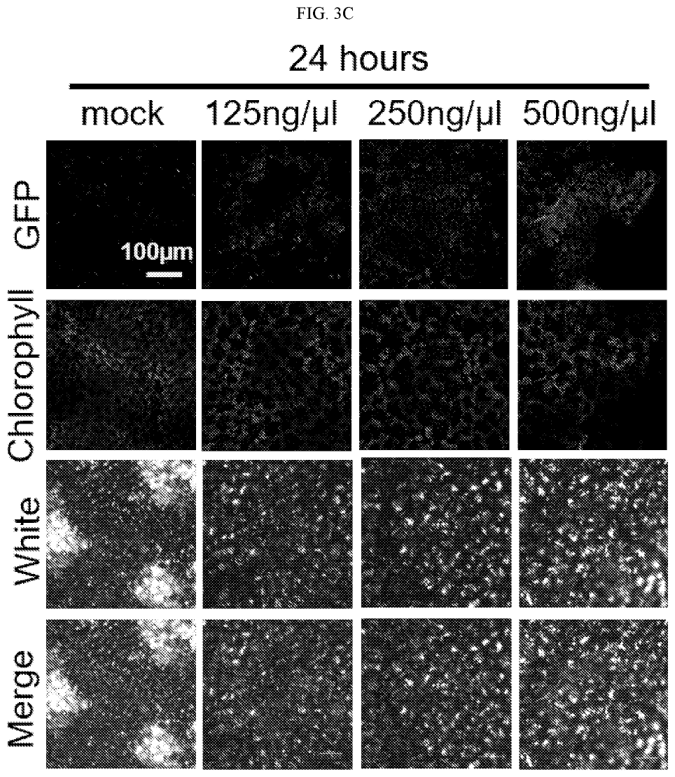

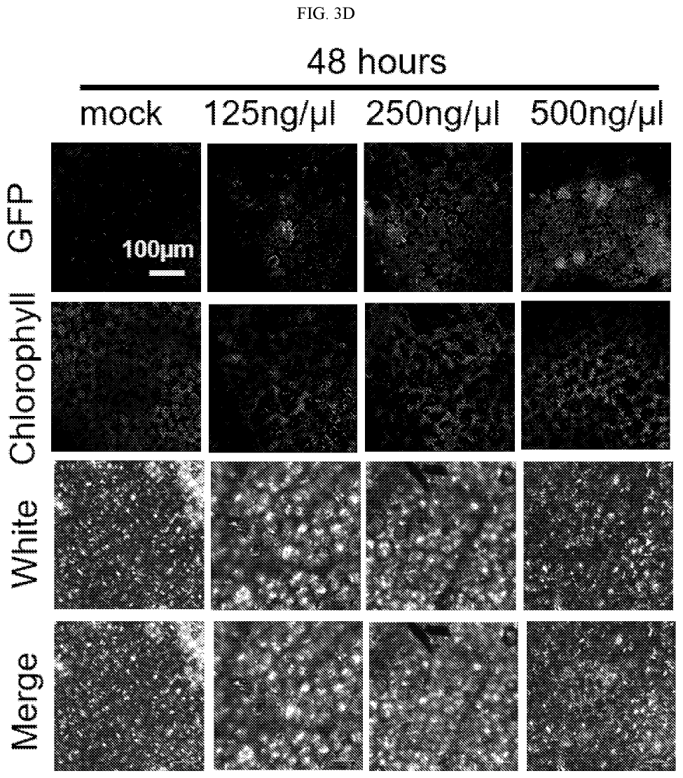

Results. The capability of SNPs to deliver DNA into intact Arabidopsis cells was examined. As shown schematically in FIG. 3B, GSH-responsive small SNPs (<60 nm) encapsulating water-soluble biomolecules (i.e. DNA and mRNA) are expected to efficiently penetrate into plant cell wall and membrane. Upon entry into the cytoplasm, the high GSH concentration (2-10 mM) is expected to induce the cleavage of the disulfide bonds and results in the release of the water-soluble biomolecule (FIG. 3B). To confirm this mechanism of action, the SNPs were loaded with linear GFP-encoding DNA fragments containing 35S promoter DNA sequences. The GFP-SNPs were then infiltrated into Arabidopsis rosette leaves with different SNP concentrations. Compared to the free GFP DNA mock control, an accumulation of GFP signals were observed in leaf cells infiltrated with GFP-SNPs at 24 hours post-infiltration, and the GFP signal intensity positively correlates with the NP concentration as much stronger signals are observed when using 500 ng/ul than 125 ng/ul NPs (FIG. 3C). Similar concentration-dependent increase in GFP intensity was found in cells at 48 hours post-infiltration (FIG. 3D). Further examination of GFP signals revealed that GFP protein was present both in cytoplasm and nucleus, suggesting the internalization of DNA-SNPs into leaf cells.

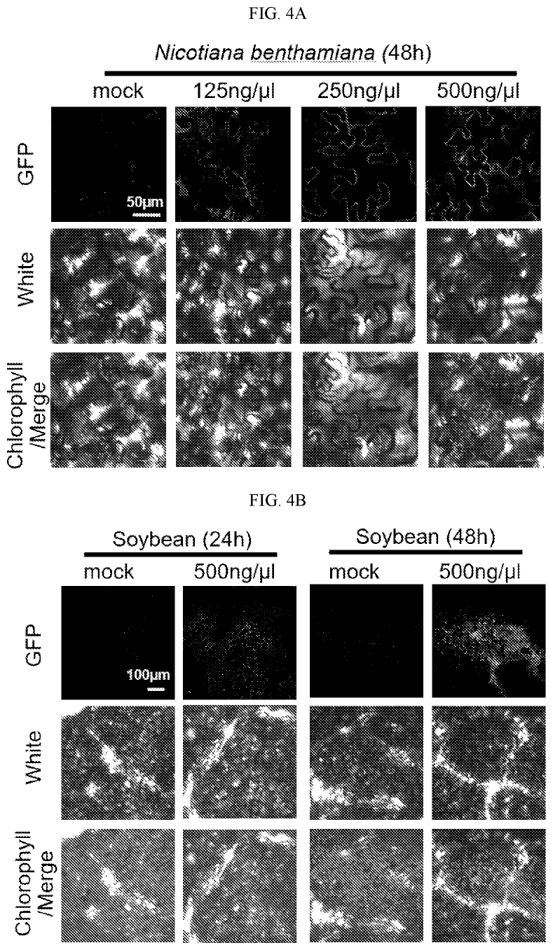

To examine whether SNP-mediated DNA delivery is dependent of specific plant species, similar experiments were performed by infiltrating GFP-SNPs into N. benthamiana mature leaves; efficient internalization of SNPs at 48 hours post-infiltration were observed (FIG. 4A). Direct comparison between different NP concentrations revealed the highest GFP fluorescence intensity at the 500 ng/ul of NP concentration (FIG. 4B). We further demonstrated that SNPs can deliver GFP DNA into soybean mature leaf illustrated by the positive GFP signals at 24 hours post-infiltration, which increase at 48 hours post-infiltration (FIG. 4B). Together, these results demonstrated that SNP is capable of delivering DNA into mature leaf cells independent of plant species.

### Example 4—SNP Infiltration into the Plant Leaf Cells—Part 2

To further confirm GFP DNA was delivered to leaf cells by SNP, A. thaliana and N. benthamiana ecotype Columbia-0 leaves were inoculated with SNP packed GFP DNA under the control of a 35S promoter following the methods of Example 3. The leaves were fixed in Tris buffer (10 mM Tris-HCl, pH 7.5, 100 mM NaCl, and 10 mM EDTA) containing 4% paraformaldehyde for 20 min. They were then washed with Tris buffer three times. The nuclei were isolated by chopping in nuclei isolation buffer (15 mM Tris-HCl, pH 7.5, 2 mM EDTA, 0.5 mM spermine, 80 mM KCl, 20 mM NaCl, and 0.1% Triton X-100). After filtering through Miracloth, the nuclei slurry was diluted with sorting buffer (100 mM Tri-HCl, pH 7.5, 50 mM KCl, 2 mM MgCl2, 0.05% Tween20, and 5% sucrose) and then dropped on poly-lysine-coated slides and allowed to air dry. The slides were fixed again with 4% paraformaldehyde in wash buffer (PBS with 0.1% Triton X-100) for 20 min and then washed three additional times with wash buffer. The nuclei were blocked with 1% BSA in wash buffer for 30 min at 37° C. and then washed three additional times with wash buffer. The nuclei were then incubated with primary antibodies with 1:200 dilution overnight at 4° C. After washing with PBS three times, the nuclei were blocked and incubated with a secondary antibody with 1:300 dilution for 2 to 4 h at 37° C. The slides were washed with PBS three times before imaging.

GFP detection using confocal laser scanning microscope. Following immunofluorescence staining of SNP-infiltrated plant cells with a GFP-antibody and corresponding secondary antibody and nuclei DNA staining with DAPI, the GFP level of the plant cells was evaluated by confocal imaging with a Nikon AIR Confocal Microscope.

Results. A. thaliana and N. benthamiana nuclei without SNP treatment did not show GFP expression, while the SNP treated leaves showed GFP expression as confirmed by anti-GFP immunostaining. As shown in FIG. 4C, the overlapping of the innate GFP signal and immunofluorescence stained GFP antibody indicated the expression of GFP after SNP transfection. Together, the results of Examples 3 and 4 demonstrated that SNP is capable of delivering DNA into mature leaf cells independent of plant species.

### Example 5—RNA Delivery into Mature Plant Cells Via SNP

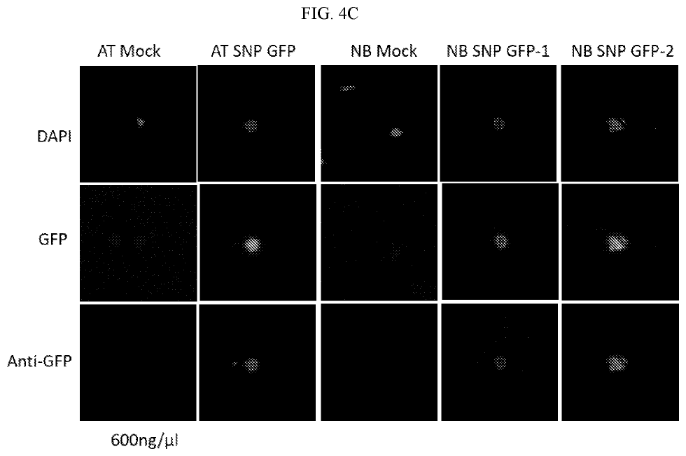

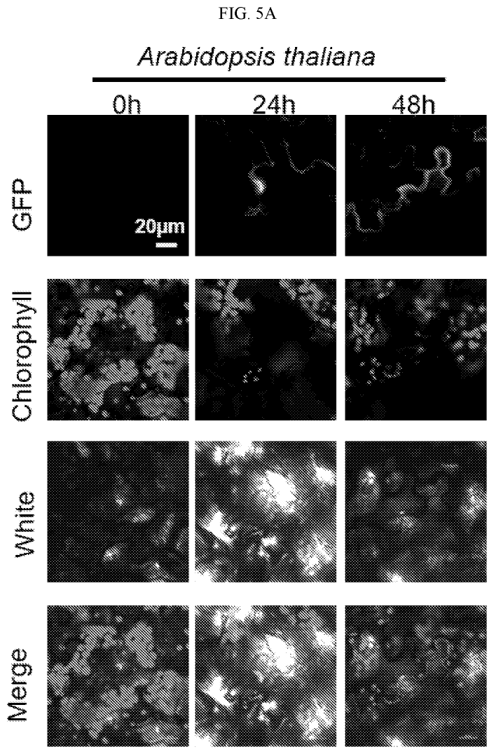

Using the techniques of Example 3, the ability of SNPs to deliver messenger RNA (mRNA) into plant cells was examined. SNPs were loaded with GFP mRNA and delivered into Arabidopsis and N. benthamiana mature leaves as before. GFP transient expression was quite efficient and was readily visible 24 hours post-infiltration with enhanced GFP signals at 48 hours in both plant species (FIGS. 5A-B), suggesting that SNP is an efficient delivery platform for mRNA transient expression in plants.

### Example 6—SNP-Mediated CRISPR-Cas9 Ribonucleoproteins (RNPs) Delivery into Plant Cells

Gene editing detection by T7EI digestion assay. Genomic DNA (˜100 ng) extracted from SNP-treated leaves was amplified for the GFP target regions by High-Fidelity DNA Polymerase Q5 (New England Biolabs, USA) (Forward: TCAGTGGAGAGGGTGAAGGT, Reverse: AAACTCAAGAAGGACCAT). The PCR products (˜1 μg) were subjected to the T7EI digestion assay following manufacturer's instruction (Intergrated DNA technologies, USA). Briefly, the PCR products were denatured at 95° C. for 10 min, followed by re-annealing with ramp PCR from 95° C. to 85° C. at −2° C./s and 85° C. to 25° C. at −0.3° C./s. These annealed PCR products were then incubated with T7 endonuclease I at 37° C. for 60 min before visualizing via 2% (w/v) agarose gel electrophoresis.

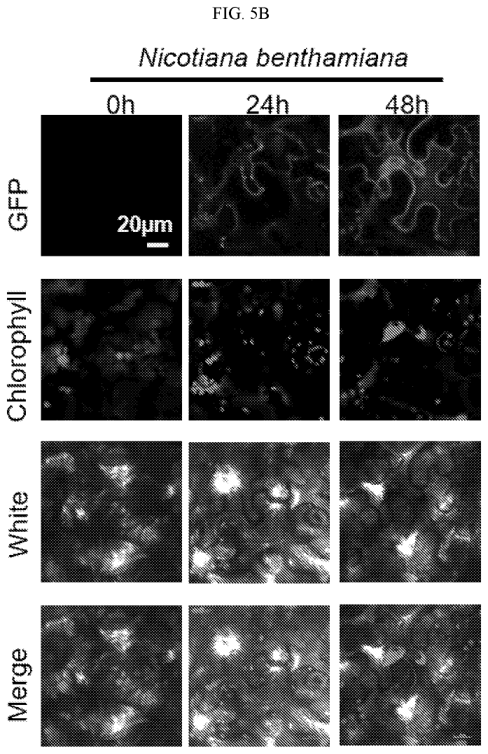

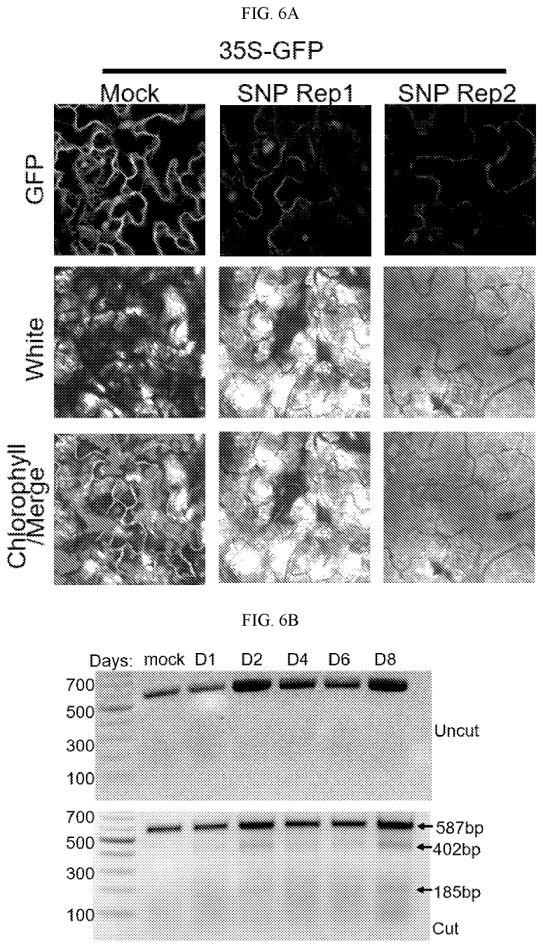

Results. To determine if SNPs of the present technology could deliver sgRNA/Cas9 ribonucleoprotein complex (RNP) to plant cells, a guide RNA (gRNA) targeting GFP sequences was synthesized by standard techniques. Nuclear localization signal (NLS)-tagged Streptococcus pyogenes Cas9 nuclease (sNLS-SpCas9-sNLS) was premixed at 1:1 molar ratio with gRNA targeting GFP and tracrRNA to form preassembled RNPs before loading into the SNPs producing nanoparticle size around 41 nm and 94% loading efficiency (Table 1). The RNP loaded SNPs were infiltrated into mature leaves of Arabidopsis plants constitutively expressing high levels of GFP under the control of 35S promoter (35S::GFP, FIG. 6A). Confocal microscopic analysis was used to determine the effectiveness and efficiency of gene editing events. Examination of GFP fluorescence intensity of confocal images of two biologically repeated experiments showed dramatically reduced GFP signals in leaf samples treated with RNP-SNPs compared to the mock control (FIG. 6A). This result demonstrates the delivery of functional RNPs by SNPs into the nucleus with an efficient editing of GFP sequences. To further confirm the genome editing events, we used the T7 endonuclease I (T7EI) mismatch assay to detect the insertion/deletion mutations of GFP gene in leaf cells treated with RNP-SNPs. The editing events were detected by the cleavage of the mismatch, resulting in two smaller bands (402 bp and 185 bp) compared to the 587 bp uncut control DNA. We detected the 402 bp and 185 bp bands of T7EI digested samples 1 day (D1) after SNP application (FIG. 5B), suggesting that RNP-mediated gene editing occurs immediately after infiltration and the editing events are stable in plant leaves even after 8 days of SNP application. Together, our results demonstrate SNP as a potent nanoplatform capable of efficient delivering functional RNPs for DNA-free plant genome editing and engineering.

### EQUIVALENTS

While certain embodiments have been illustrated and described, a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Likewise, the use of the terms “comprising,” “including,” “containing,” etc. shall be understood to disclose embodiments using the terms “consisting essentially of” and “consisting of.” The phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A nanoparticle comprising:
a silica network comprising crosslinked polysiloxanes, wherein the crosslinks comprise disulfide linkages, and the nanoparticle has a surface bearing charged functional groups and a surface potential of either less than −30 mV or greater than +30 mV, and wherein the nanoparticle has an average diameter of 20 nm to 60 nm.

2. The nanoparticle of claim 1, wherein the polysiloxanes comprise a plurality of siloxy subunits having the structure 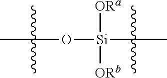
or the structure 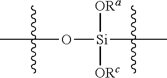
wherein R^{a} and R^{b} at each occurrence in the polysiloxane are independently selected from a bond to a Si of another polysiloxane chain or C₁₋₆ alkyl groups, and R^{c} is selected from C₂₋₆ alkenyl groups.

3. The nanoparticle of claim 2, wherein the plurality of siloxy subunits are derived from tetraethoxysilane or triethoxyvinylsilane.

4. The nanoparticle of claim 2, wherein the polysiloxanes comprising the plurality of siloxy subunits having the structure 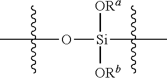
comprise a first portion of siloxy subunits wherein R^{a} and R^{b} are independently selected from C₁₋₆ alkyl groups, and a second portion of siloxy subunits wherein one of R^{a} and R^{b} is independently selected from C₁₋₆ alkyl groups at each occurrence, and one of R^{a} and R^{b} is a bond to a Si of another polysiloxane chain.

5. The nanoparticle of claim 1, wherein the polysiloxanes further comprise a plurality of siloxy subunits bearing imidazolyl groups.

6. The nanoparticle of claim 5, wherein the plurality of siloxy subunits bearing imidazolyl groups are derived from TESPIC.

7. The nanoparticle of claim 1, wherein the polysiloxanes comprise a plurality of crosslinking subunits having the structure 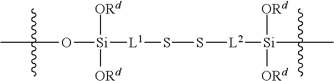
wherein
L¹ and L² at each occurrence in the polysiloxanes are independently a C₁₋₆ alkylene group;
R^{d} at each occurrence in the polysiloxanes is the same or different and is independently selected from a bond to a Si of another polysiloxane chain or C₁₋₆ alkyl groups.

8. The nanoparticle of claim 1, wherein the polysiloxanes comprise a plurality of siloxy subunits having the structure 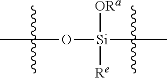
wherein R^{a} at each occurrence in the polysiloxane is a bond to Si from another polysiloxane chain or a C₁₋₆ alkyl group, and R at each occurrence is a C₁₋₆ alkyl group substituted with a charged functional group.

9. The nanoparticle of claim 1, wherein the charged functional groups comprise ionizable functional groups selected from amine, amidine, guanidine, pyridinyl or combinations of two or more thereof.

10. The nanoparticle of claim 9, wherein R^{e} is an amino-(C₂-4 alkylene) group.

11. The nanoparticle of claim 1, wherein the surface comprises a cationic polymer or a cell penetrating peptide bearing the charged functional groups and the cationic polymer is selected from the group consisting of polyethyleneimine (PEI), polylysine, polyarginine, and polyamidoamine (PAMAM).

12. The nanoparticle of claim 1, wherein the charged functional groups comprise ionizable functional groups selected from carboxyl, sulfonyl, sulfate, phosphate, or combinations thereof.

13. The nanoparticle of claim 8, wherein R^{e} is a carboxyl-(C₂₋₄ alkyl) group.

14. The nanoparticle of claim 1, wherein the surface comprises an anionic polymer bearing the charged functional groups and is selected from the group consisting of poly(glutamic acid) and poly(acrylic acid).

15. The nanoparticle of claim 1, wherein the charged functional groups are positively charged groups and the surface potential is greater than +30 mV.

16. The nanoparticle of claim 1, wherein the charged functional groups are negatively charged groups and the surface potential is less than −30 mV.

17. The nanoparticle of claim 1 wherein the average diameter is 30 nm to 50 nm.

18. The nanoparticle of claim 1, further comprising a water-soluble biomolecule non-covalently bound to the nanoparticle.

19. The nanoparticle of claim 18, wherein the water-soluble biomolecule is selected from the group consisting of a polynucleic acid, polypeptide, and a polynucleic acid/polypeptide complex.

20. The nanoparticle of claim 18, wherein the water-soluble biomolecule is DNA, RNA, or a ribonucleoprotein complex (RNP).

21. The nanoparticle of claim 18, wherein the water-soluble biomolecule is selected from RNP, plasmid DNA (pDNA), single-stranded donor oligonucleotide (ssODN), complementary (cDNA), messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof.

22. The nanoparticle of claim 18, wherein the water-soluble biomolecule is Cas9 RNP or RNP+ssODN.

23. The nanoparticle of claim 0, wherein the water-soluble biomolecule is a polypeptide.

24. A method of delivering a water-soluble biomolecule into a plant cell comprising exposing the plant cell to an effective amount of a nanoparticle of claim 18.
